(19) United States
(12) Patent Application Publication
(10) Pub. No.: US 2009/0105687 A1
(43) Pub. Date: Apr. 23, 2009

(54) SCORING CATHETER WITH DRUG DELIVERY MEMBRANE

(75) Inventors: Robert K. Deckman, (US); Brian Placek, (US); Michael A. Munrow, (US); Craig Gerbi, (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: AngioScore, Inc., Fremont, CA (US)

(21) Appl. No.: 12/239,636

(22) Filed: Sep. 26, 2008

Related U.S. Application Data
(60) Provisional application No. 60/977,885, filed on Oct. 5, 2007.

(51) Int. Cl. A61M 25/10; A61K 31/404; A61K 31/235; A61K 31/07; A61K 31/7088; A61K 48/00; A61K 35/12; A61K 39/395; A61K 31/337; A61K 31/335; A61P 9/00

(52) U.S. Cl. 604/509; 604/103.02; 514/415; 514/568; 514/725; 514/44; 424/93.7; 424/93.21; 424/141.1; 514/449

(57) ABSTRACT

Angioplasty and other dilatation devices are provided with scoring elements. The device additionally incorporates a separate drug application membrane which is used to deliver drugs and other active substances to a body lumen, typically a blood vessel. The device functions to release drug into a region of the luminal wall as the scoring structure is radially expanded into the lumen wall.

### CROSS-REFERENCE TO RELATED APPLICATIONS

This application claims the benefit of provisional application No. 60/977,885 (Attorney Docket No. 021770-001200US), filed on Oct. 5, 2007, the full disclosure of which is incorporated herein by reference.

### BACKGROUND OF THE INVENTION

1. Field of the Invention

The present invention relates to the field of medical devices, more specifically medical devices intended to treat stenoses in the vascular system.

Balloon dilatation (angioplasty) is a common medical procedure used revascularize or unclog stenotic vessels, most commonly atherosclerotic plaque narrowed arteries. The angioplasty procedure works by threading a catheter, tipped with a deflated dilatation balloon, through the vascular system to the target stenotic site. Once at the appropriate site, the balloon is inflated. The inflated balloon applies radial pressure to the inner wall of the vessel. This helps redistribute the plaque to a more favorable (less blocking) configuration, and widens the stenosed region to enable better blood flow.

Unfortunately, in many cases after balloon dilatation, the plaque and the blood vessel walls eventually redistribute to an unfavorable configuration again, often causing vessel reocclusion. To prevent this, the balloon dilatation procedure is often followed immediately by a stenting procedure. In a stenting procedure, a tubular scaffold, called a stent, is placed inside the vessel to help keep the vessel open, and maintain vessel patency. In addition to physical scaffolding, many stents also elute drugs to help prevent restenosis. Such stents are called drug eluting stents or DES.

Stenting itself is not perfect, however, and reocclusion still occurs at too high a rate. This is due to a number of factors. Although, as previously discussed, many stents elute drugs to help prevent restenosis, for the drugs to be effective; they must be delivered to the proper site. However if the stenotic vessel has not been properly widened by the angioplasty balloon, the stent may, in fact, not be properly positioned.

Drug eluting stents (DES) also have other issues as well, and are not suitable for every patient. Patients undergoing DES implantation are often kept under a regimen of anti-coagulant therapy for an extended period of time to minimize risk of late thrombosis. However such anticoagulants may cause excessive bleeding and are not recommended for all patients.

Other problems that can occur with stent placement include the presence of gaps between the stent and the vessel wall, and calcified areas of the vessel plaque or wall that are resistant to balloon dilation.

Conventional balloon angioplasty suffers from a number of other shortcomings as well. In some cases, due to overly aggressive balloon dilation, the balloon dilatation procedure stretches the diseased vessel beyond its elastic limits, causing vessel damage. These damaged vessel walls have a higher risk of restenoses. In other cases, slippage of the balloon during the dilatation procedure may occur. This may result in injury to the vessel wall surrounding the treated lesion.

As a result, some vascular occlusions are difficult to treat by conventional angioplasty balloons. Some of these difficult situations include in-stent restenosis (i.e. a secondary restenosis occurring after the original restenosis was treated by angioplasty and a stent). In-stent restenoses is difficult to treat by angioplasty balloons because the stent resists expansion, and the angioplasty balloon thus tends to slip away from the target zone during the inflation process. Another difficult to treat problem is caused by fibrotic lesions, which are relatively resistant to stretching. When angioplasty balloons encounter such fibrotic lesions, they again tend to slip away from the proper target zone on inflation. Unfortunately, many long lesions have fibrotic sections, and as a result are difficult to treat using conventional angioplasty balloons.

One way to overcome these issues is to associate blades or scoring elements with the angioplasty balloon. The blades or scoring elements both help prevent the balloon from slipping out of the proper position, and can also cut through some of the more resistant portions of the vessel lining. This prevents slippage, and allows the intended vessel dilation to proceed.

A second advantage to using blades or scoring elements with angioplasty balloons is that blood vessel walls, particularly plaque occluded walls, tend to tear in an uncontrollable manner during balloon inflation. Cutting blades can help minimize this damage, because the cutting and tearing, instead of being uncontrolled, can now be controlled and limited. Only the minimal amount of cutting or scoring necessary to accomplish the dilation procedure needs to be done, and this cutting or scoring can be confined to an identified and localized area of the vessel.

While the use of angioplasty balloons having cutting blades has proved to be a significant advantage under many circumstances, the present cutting balloon designs and methods continue to suffer from shortcomings.

As previously discussed, use of drugs or therapeutic agents on cut, scored, or damaged section of blood vessel linings can help reduce restenosis and improve outcomes. However many of these drugs should ideally be applied directly to the site of vessel damage, rather than systemically. Since scoring results in precisely localized areas of vessel damage, ideally drugs or other therapeutic agents should be brought exactly to these localized areas of damage, and used to mitigate the effects of this damage. Such precise delivery would both allow a higher concentration of drugs and therapeutic agents to be administered, and would also decrease any unwanted side reactions between such drugs and non-target tissues. To that end, the coating of scoring elements with anti-proliferative and other agents has been proposed in commonly owned US 2006/0259005 A1. While a significant improvement, the need to coat or otherwise sequester drugs on a scoring element can be technically challenging, may limit the amount of drug that can be delivered, and makes inventory maintenance of different drugs and different dosages problematic.

Another problem with coating the scoring elements is that drugs coated on the outside of catheters tends to diffuse away before reaching the proper site. A further problem is that it will not always be possible to adhere or sequester enough drug on the scoring elements to be effective.

For these reasons, it would be desirable to provide methods and systems which could utilize “drug-free” scoring and cutting structures for delivering therapeutic agents to blood vessels and other body lumens. Such methods and systems could disrupt vascular and luminal occlusions by scoring and cutting structures, while permitting simultaneous delivery of therapeutic agents to the blood vessel without requiring that the scoring/cutting elements themselves to be modified. In particular, methods and systems that can work together with the cutting element in order to provide for the delivery of the therapeutic agents, preferably at a variety of dosages and under a variety of conditions. At least some of these objectives will be met by the inventions described herein below.

2. Description of the Background Art

The following U.S. patents and printed publication relate to cutting balloons and balloon structures: U.S. Pat. Nos. 6,450,988; 6,425,882; 6,394,995; 6,355,013; 6,245,040; 6,210,392; 6,190,356; 6,129,706; 6,123,718; 5,891,090; 5,797,935; 5,779,698; 5,735,816; 5,624,433; 5,616,149; 5,545,132; 5,470,314; 5,320,634; 5,221,261; 5,196,024; and Published U.S. Pat. Apps. 2006/0259005 and 2003/0032973. Other U.S. patents of interest include U.S. Pat. Nos. 6,454,775; 5,100,423, 4,998,539; 4,969,458; and 4,921,984. The following patents describe drug delivery catheters having needle based delivery mechanisms: U.S. Pat. No. 4,578,061 describes needle injection catheters having deflectable, axially advanceable needles. U.S. Pat. No. 5,538,504 describes a needle injection catheter having a transversely oriented needle that is laterally advanced by a balloon driver. Also of interest are U.S. Pat. Nos. 6,319,230; 6,283,951; 6,283,947; 6,004,295; 5,419,777; and 5,354,279. Drug coated stents and angioplasty balloons are described in numerous patents and published applications including U.S. Pat. Nos. 6,280,411; 6,656,156; 6,682,545; and Publication Nos. US2004/0193257; US2004/0208985; and US2005/0033417.

Patents and published applications relating to angioplasty balloons with drug coatings and optionally having cutting blades include U.S. Pat. Nos. 5,102,402; 5,120,322; 5,304,121; 5,868,719; 6,929,320; 6,364,856; 6,592,548; 7,060,051; 2004/127475A1; 2004/143287A1; 2005/288629A1; 2006/004323A1; 2006/020243A1; 2006/129093A1; 2006/247674A1; 20056/259062A1; WO95/03083A1; WO99/16500A2; and EP 1007135B1.

A number of different anti-slip, cutting, and scoring configurations have been proposed in the art. U.S. Pat. No. 5,320,634 describes the addition of cutting blades to the balloon. The blades can cut through fibrotic lesions as the balloon is inflated. U.S. Pat. No. 5,616,149 describes a similar method of attaching sharp cutting edges to the balloon. U.S. Patent Publication 2003/0032973 describes a stent-like structure having non-axial grips for securing an angioplasty balloon during inflation. U.S. Pat. No. 6,129,706 describes a balloon catheter having anti-slip bumps on its outer surface. U.S. Pat. No. 6,394,995 describes a method of reducing the balloon profile to allow crossing of tight lesions. U.S. Patent Publication 2003/0153870 describes a balloon angioplasty catheter having flexible elongate elements that create longitudinal channels in a stenosis or other occluded region.

Drug delivery balloons and membranes are also described in U.S. Pat. Nos. 5,102,402; 5,120,322; 5,304,121; 5,383,928; 5,707,385; 5,868,719; 6,471,979; 6,565,528; and 7,011,654; published U.S. Pat. Apps 2004/0127475; 2004/0143287; 2005/0288629; 2006/0004323; 2006/0020243; 2005/0083768; 2006/0112536; and 2006/0259062. US Pat. App 2006/0129093 describes a multiple inflatable balloon catheter where at least one of the balloons may have at least one scoring blade, and the exterior surface of at least one balloon may contain a drug or therapeutic agent. U.S. Pat. No. 6,939,320 describes an elastic catheter sheath containing perforations that open as the substrate inside the catheter sheath expands, releasing drugs that have been coated on the substrate. U.S. Pat. No. 6,364,856 describes an expandable catheter covered with a sponge coating containing multiple voids that can release drugs or biologically active material upon expansion. U.S. Pat. No. 6,656,156 describes a balloon catheter with a first balloon coating containing a drug, and a second coating covering the first drug coating that fractures when the balloon expands, releasing drug.

### BRIEF SUMMARY OF THE INVENTION

The present invention provides methods and apparatus for delivering substances to luminal sites, and in particular for delivering active substances such as anti-proliferative and anti-hyperplasia substances (drugs) to diseased sites in a patient's vascular system, such as sites of thrombosis and plaque in a patient's arteries. Methods for delivering active substances to a luminal site (i.e. a target site such as a stenosed region of an artery) comprise positioning a scoring structure within the body lumen and advancing the scoring structure to score a wall of the body lumen. Drug delivery, which can be focused on the scored region(s) of the lumen as desired, is provided by a drug delivery membrane, distinct from the other catheter components, placed either over the scoring element (as an envelope over all or portion of the scoring structure) or behind the scoring structure (between a scoring structure and a balloon. This drug delivery membrane system works in conjunction with the scoring element to deliver an active substance (drug) to the luminal site. The active substance can be delivered to the surface of the luminal wall region before scoring, during scoring, or after scoring.

Although the present drug delivery membrane devices and methods of the present invention can work with a wide variety of scoring catheter designs, it will be particularly useful to employ the scoring element construction previously described in commonly owned application Ser. No. 10/917,902, the full disclosure of which is incorporated herein by reference.

In an exemplary vascular use, the active substance can be released to locations in or beneath the intimal layer of the vessel wall, typically to a depth in the range from 0.001 mm to 1 mm, usually from 0.01 mm to 0.1 mm. In the case of treatment of arterial sites, simultaneous scoring of the region, occlusive material, and/or the wall facilities can not only deliver the drug to regions within the thrombus or plaque, it can further deliver the drug into the intimal and subintimal layers surrounding the blood vessel.

In addition to treatment of blood vessels, the methods and systems of the present invention can be used to treat a variety of other body lumens, including vein grafts and synthetic grafts, as well as lumens of the respiratory, urinary, reproductive and digestive systems, and the like.

The benefits of combining scoring with targeted drug delivery are substantial. With scoring alone, the damaged vessel regions would be exposed to the vascular environment for some period of time before receiving any therapeutic agents. Delivery of the agents with the scoring catheter allows the damaged vessel region to receive targeted therapeutic agents either immediately before, during, or immediately after any damage or insult. Since many inflammatory and coagulation processes are cascade processes in which a small initial signal, such as release of naturally occurring tissue thromboplastin, triggers a biochemical cascade of often unwanted reactions, the advantages of almost instantly preventing or stopping such unwanted biochemical cascade process with properly timed and targeted therapeutic agents are substantial.

In addition to the benefits of simultaneous or near simultaneous drug delivery, the use of a separate membrane as a drug reservoir has many advantages. The amount of drug can be much greater than that which can be coated on the scoring elements and/or the balloon surface. The membrane can be used with catheters which themselves have scoring element balloons or coated with drugs, thus allowing different drugs to be released in combination or even greater amounts of drugs to be released.

### BRIEF DESCRIPTION OF THE DRAWINGS

FIGS. 1A and 1B are schematic illustrations of the drug delivery membrane mounted exterior to the scoring elements, in accordance with an embodiment of the invention.

FIGS. 2A and 2B are schematic illustrations showing drug delivery membranes, mounted exterior to the scoring elements, delivering therapeutic agents to scored regions of a body lumen.

FIGS. 3A, 3B and 3C are schematic illustrations showing an embodiment of the invention in which the drug delivery membrane is mounted interior to the scoring elements, optionally perforated with pores, and optionally attached to the scoring elements at multiple locations.

FIGS. 4A and 4B are schematic illustrations showing how the device of FIG. 3 would simultaneously score and deliver therapeutic agents to a plaque occluded artery.

FIGS. 5A, 5B, and 5C are schematic illustrations showing an embodiment of the invention in which the drug delivery membrane is mounted exterior to the scoring elements, and is perforated with pores located near the scoring elements, and optionally is attached to the scoring elements at multiple locations.

FIGS. 6A, 6B, and 6C are schematic illustrations showing an embodiment of the invention in which the drug delivery membrane is non-elastic, and is mounted interior to the scoring elements. Here the therapeutic agents are stored in folds (pockets) in the drug delivery membrane in hydrogel, polymer embedded, or other meta-soluble state, and are released as the scoring elements score the plaque or other body lumen lining.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

Embodiments of the present invention relate to devices for revascularization of stenotic vessels, and more specifically to a catheter with blood vessel dilation elements, as well as additional lumen scoring and membrane drug delivery elements.

Although ideally intended for treating coronary arteries that have been occluded by plaque, the invention will also find use in treating other body lumens, such as vein and synthetic grafts, as well as lumens of the respiratory, urinary, reproductive and digestive systems, and the like, for other conditions such as lesions or tumors or some types of cancer or other local disorders.

Throughout this disclosure, the substances that can be delivered by the drug delivery membranes of the present invention are generally termed drugs, active agents, or substances. In fact a wide variety of different chemical substances can be delivered, and use of specific examples, such as “drugs” or nomenclature, such as “drug delivery membrane”, is not intended to limit the range of substances that can be delivered by the invention. In certain situations, it will also be useful to introduce non-drug, non-active agents, and non-therapeutic agents, such as contrast agents to help visualize the extent of vascular damage. In other situations, it may be useful to introduce biological agents, such as viruses or living cells (e.g. stem cells) to help promote vessel repair.

A more extended discussion of the types of drugs, active agents, and substances that may be administered, as well as a more extended discussion of the chemical makeup of the drug delivery membrane, will be given at the end of this section. First, however, the structural and mechanical aspects of the device will be discussed.

The blood vessel dilatation device may comprise a conventional dilatation balloon such as a polymeric balloon. The optional lumen scoring elements may comprise one or more scoring blades, wires, or structures arranged in a linear, spiral, helical, or other configuration. In some embodiments, these scoring elements are mounted on the balloon catheter. The apparatus additionally comprises a drug delivery membrane system. Drugs or active agents may be coated or loaded onto or into the membrane, or alternatively the membrane may be used to control the delivery of an active substance to be released into the blood vessel wall.

Although scoring balloon catheters are frequently used as examples herein, the present membrane system drug delivery invention is not limited to balloon catheters with scoring elements. The present drug delivery membranes may be used with POBA (plain old conventional balloon angioplasty) devices, as well as other catheters with a variety of different expandable elements. Although these expandable elements may comprise expandable balloons, they may also include other deployment means including expandable polymers, memory wire, heating expansion elements, electrical expansion elements, chemical expansion elements, or mechanical expansion elements.

The drug delivery membrane system of the present invention will often be administered at the time of the initial scoring of the body lumen, although it also may be used before or after as needed.

The scoring element(s) of the present invention are typically positioned using an intravascular or other intraluminal catheter. The catheter will carry one or more scoring elements at or near its distal end. In the case of blood vessels, the catheter is typically introduced over a guidewire in a conventional manner, e.g., through the femoral artery to reach the coronary arteries, or through a sheath in case of peripheral arteries.

The scoring element(s) may be advanced to score a target region of a body lumen (such as a plaque stenosis in an artery) by radially advancing the scoring elements into the lesion and the luminal wall. As previously discussed, such radial expansion is typically achieved using an expandable inner shell, such as an inflatable balloon carried by the catheter. However alternatively, the radial expansion can be achieved using self-expanding materials such as nitinol or expandable geometries using other materials (such as stainless steel). For brevity, the examples discussed here focus on inflatable balloons; however this use is not intended to limit the scope of the invention in any way.

The scoring elements of the present art may have any of the geometries previously used in scoring devices, including various linear blade geometries. In some cases, however, the scoring elements will comprise one or more resilient and voids elements having helical geometries, as taught by co-pending patent application Ser. Nos. 10/631,499 (Attorney Docket No. 021770-000100US), filed on Jul. 30, 2003; Ser. No. 10/810,330 (Attorney Docket No. 021770-000120US), filed on Mar. 25, 2004; and Ser. No. 10/917,917 (Attorney Docket No. 021770-000130US), filed on Aug. 13, 2004, assigned to the assignee of the present application, the full disclosures of which are incorporated herein by reference.

The scoring structures used in the present disclosure will typically consist of both scoring elements and voids. Voids (absence of space) play an important role in scoring structures because voids focus pressure on the cutting or scoring elements. Without voids, the force of the solid elements of the scoring structure will become evenly distributed throughout the inner lumen of the body surface, and no effective scoring will result. Typically greater than 90% of the scoring structure will actually consist of voids.

Regardless of the geometry of the scoring elements, once positioned at the correct target zone, the scoring elements will usually be advanced (expanded) in an outward radial manner by expanding a scoring element support shell. Often this will be done by inflating a balloon which carries at least one scoring element. In this way, the outward edge(s) of the scoring element can engage and penetrate the luminal wall and/or the occlusive or other material (such as plaque), which covers at least a portion of the luminal wall.

As previously discussed, alternatively, the radial expansion can be achieved using self-expanding materials such as nickel titanium alloys or expandable geometries using other materials (such as stainless steel). The scoring elements can also be expanded by other means such as temperature controlled structured materials (e.g. heat memory alloys), or mechanical expansion means such as internal sliders with an increased diameter.

Usually the scoring and drug delivery membrane system of the present invention will be delivered to its target zone using a catheter. Catheters of the present invention comprise a catheter body having a proximal end (the operator end that may extend outside the body) and a distal end (the nose end of the catheter). Typically the scoring element or elements will be disposed near the distal end.

The catheter will normally comprise an internal expandable element, such as a pressure expandable elastic balloon. Although this balloon itself may comprise a membrane or wall, the drug delivery membranes of the present invention are separate and distinct from this elastic balloon membrane or wall.

In contrast to the elastic, pressure expandable balloon membrane/wall material, the drug delivery membrane, which will typically surround the balloon material, does not itself necessarily need to be either elastic or pressure expandable, although it can be. The drug delivery membrane simply has to control the delivery of drugs or active agents, and the primary pressure and mechanical load-bearing functions will typically be delegated to the either the dilation balloon membrane/wall or the scoring elements. This separation of functions allows the drug delivery membrane to serve its primary function—delivering drugs. For example, the membrane may be non-distensible (i.e., have an elastic modulus above 5 GPA, often above 10 GPA), semi-compliant (between 1 GPA and 5 GPA), or compliant (below 1 GPA).

Depending upon the desired configuration, the scoring element or elements may be mounted either in the space between the pressure expandable elastic dilation balloon and the drug delivery membrane, or the scoring element may be mounted outside of the drug delivery membrane, thus surrounding both the pressure expandable elastic balloon and the drug delivery membrane.

The drug delivery membrane will normally work in conjunction with the action of the scoring element to deliver a drug or active substance to a luminal wall scored or cut by the scoring element. The active substance may be provided by the action of the drug delivery membrane and the scoring element in a variety of ways.

As a first example, the active substance is either deposited on the surface of the drug delivery membrane, or permeates the matrix of the drug delivery membrane. The drug is delivered to the scored region of the body lumen by either direct contact or diffusion.

In this embodiment, the drug delivery membrane may be mounted outside of the scoring element, and the drug delivery membrane is pressed into the lumen wall by the action of the scoring element. The drug delivery membrane acts to allow the drug or active agents to be administered to both the areas of the luminal wall scored by the scoring element (highest dose, since it is being mechanically forced into the luminal wall), and the areas of the luminal wall that are not scored by the scoring element (lower dose, since the contact pressure will be less here).

The drug may be coated over at least a portion of an exposed surface of the drug delivery membrane, typically by dipping, spraying, painting, plasma deposition, electroplating, centrifuge systems or the like. Alternatively however, the active substance may be incorporated in a polymeric membrane matrix carrier. Such polymeric carriers and matrixes will be discussed in more detail in the chemical portion of this disclosure.

The term “permeating a membrane matrix” is used to describe the concept that the basic material that the drug delivery membrane is made from (usually a cross-linked polymer) has an ability to intercalate drug and other molecules at the molecular level (e.g. between the natural set of molecular void spaces. Examples of such natural molecular void spaces include the space in between the cross-linked chains that link the membrane matrix polymer together).

This permeable membrane matrix concept is different from the alternative concept of membranes that have been rendered permeable through the introduction of pin-holes or pores. Such pin-holes and pores allow drug molecules to flow directly through the membrane without becoming entangled with the basic membrane polymeric structure.

Drugs will normally elute from a membrane matrix by a process of diffusion. By contrast, drugs may be emitted from membrane pores or pin-holes by active flow through a pressure gradient.

In an alternative embodiment, the scoring element may be mounted in-between the inner expandable elastic balloon element, and the outer drug delivery membrane. The drug delivery membrane may additionally have selected points of attachment where it attaches to either the scoring element or the underlying elastic balloon element. The scoring element may additionally contain optional serrations, protrusions, channels, or openings that act to selectively penetrate (poke through) various regions of the drug delivery membrane creating pores. In this configuration, these pores, serrations, protrusions, channels or openings work in conjunction with the drug delivery membrane to allow drug on the inside of the drug delivery membrane to flow through the membrane and penetrate into the scored regions of the lumen.

In some embodiments, the drug may be injected into a drug delivery membrane reservoir (such as the area between the elastic balloon element and the drug delivery membrane) at the time of use, and may then be released from this reservoir via pores, pin-holes, or other mechanism. This injected drug could come from either a catheter drug reservoir, or alternatively could come from a tube connecting the local drug delivery membrane drug reservoir to a main drug reservoir located outside of the catheter.

Alternatively, the scoring element may again be mounted outside of the drug delivery membrane. In this example, the drug delivery membrane may be non-elastic, and may be folded in a way so as to create drug containing membrane wrinkles or pockets that are folded around the scoring element. As the inner elastic balloon expands, it will press both the scoring element and the drug containing drug delivery membrane wrinkles or pockets up against the scored areas of the lumen surface

As previously discussed, scoring elements used with the drug delivery membranes of the present disclosure may have any conventional geometry, generally as described above, including linear, helical, or other geometries. In the exemplary embodiments, the scoring elements will be formed as at least a portion of a resilient cage which surrounds an expandable shell carried by the treatment catheter. The resilient cage will have a structure which expands with shell expansion and collapses over the shell, e.g., helping to deflate a balloon which carries the cage.

Although normally the drug delivery membrane will be distinct from both the scoring elements and the underlying expandable elastic balloon element, in some embodiments of the device, there may be certain areas of attachment between these elements. As an example, in some configurations, it may be useful to have selected points or linear regains of attachment between the drug delivery membrane, and the elastic balloon element. These regions of attachment will help control the shape of the drug delivery membrane, and ensure that selected regions of the drug delivery membrane (such as pores or pin-holes) line up properly with selected regions on the scoring device. This in turn will help insure that the drug is delivered to the desired locations.

Although, in many configurations, the drug delivery membrane will be an intact membrane that has no large (artificially induced) pores, holes, pinholes, or openings. In other cases, the drug delivery membrane may be made of a material, such as Gore-Tex® membrane that has naturally occurring pores, such as the membranes previously described in U.S. Pat. No. 5,213,576.

In still other embodiments, however, the drug delivery membrane may contain small and deliberately placed pores, holes or slits though which drug may flow. Here “deliberately placed” means that such pores, holes, or slits are not a natural part of the membrane microstructure (such as is the case with Gore-Tex membranes), but rather have been deliberately placed at defined positions in the membrane during the manufacturing process. Such pores, holes and slits will be referred to generically as “pores.” They can be created by a variety of different means including mechanical or laser drilling, chemical or photochemical etching, radiation etching, or other process.

If such deliberately placed pores, holes or slits are desired, it may additionally be advantageous to arrange these holes and slits to correspond with the scoring elements, so as to produce drug flow close to the regions of the lumen scored by the scoring elements. To facilitate this process, the drug delivery membrane may occasionally be attached to selected regions either the scoring device and/or the underlying elastic expansion balloon element to ensure proper alignment of drug delivery holes or pores and scoring elements as the underlying expansion balloon expands the device, and also to ensure that the device refolds into a smaller shape again to facilitate eventual withdrawal from a body lumen, if desired.

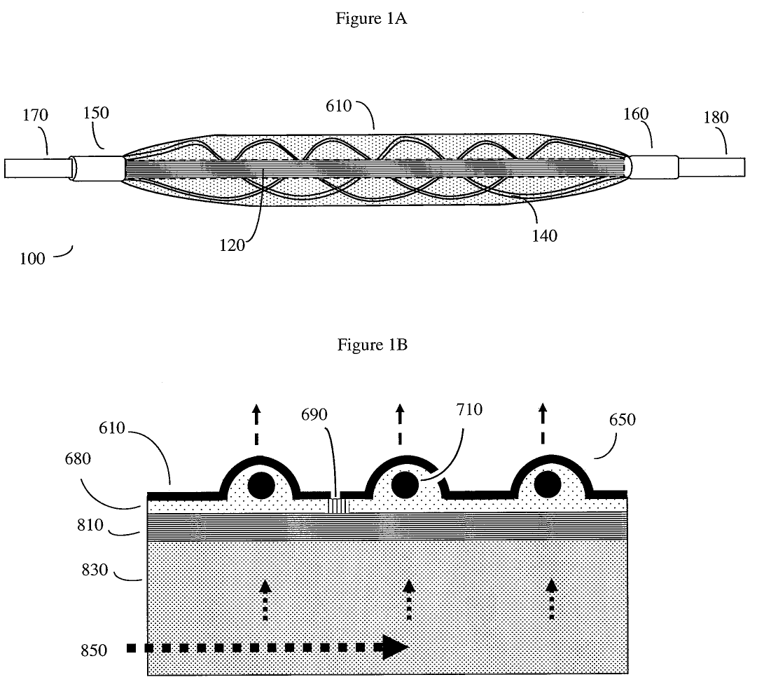

Reference is now made to FIGS. 1A and 1B, which are schematic illustrations of a combination scoring and drug delivery catheter device (100) in accordance with embodiments of the invention.

FIG. 1A shows an overview of the combination scoring and drug delivery catheter device (100). In this embodiment, the scoring/drug delivery catheter device (100) includes a dilatation balloon (120) (here shown in the non-inflated state), which may be any conventional angioplasty balloon such as commonly used by interventional cardiologists or radiologists. Here a helical or spiral scoring unit (140) is mounted over (or attached to) dilatation balloon (120). The compliance of the balloon and the scoring element(s) should be chosen to assure uniform expansion of the balloon. In particular it will be important to select materials to avoid non-uniform “end greater than middle” expansion (commonly referred to as “dog-boning”) as the combined structure expands within a lesion.

Here again choice of materials and structures is important. If a compliant or a semi-compliant balloon is used, and the compliance of the scoring element is not properly matched to comply with the properties of the balloon, the expansion of the balloon-scoring element system will not be uniform. This non-uniformity may impair the efficacy of the scoring catheter and, in some cases, may result in poor performance. For example, under given pressure, certain parts of the balloon will be able to expand while other parts will be constrained by excessive resistance of the scoring elements.

Scoring unit (140) in many embodiments is made of nitinol and may optionally have a helical structure. However scoring unit (140) may be made of other metals such stainless steel, cobalt-chromium alloy, titanium, and the like. Alternatively, scoring unit (140) may be a polymeric material, or may be made of another elastic material. Scoring unit (140) may be attached at its proximal and distal ends to the proximal end (170) and distal end (180) of dilatation balloon (120) (here assumed to be mounted on a catheter with the same orientation). Alternatively, scoring unit (140) may be attached to the distal end and/or the proximal end of dilatation balloon (120) by collar-like attachment elements (150) and (160). Spring or other compliant elements may be alternatively or additionally provided as part of the attachment elements to accommodate shortening of the scoring unit as it is expanded.

In this configuration, scoring unit (140) is surrounded by drug delivery membrane (610) (here shown in a more inflated state to clearly distinguish it from the balloon (120). Drug delivery membrane (610) does not have to be particularly load bearing, and thus will often be more pliable than the membrane (810) that forms the wall of dilatation balloon (120).

Dilation balloon (120) is load bearing, and thus usually will have walls (810) that are thick enough and semi-rigid enough (when inflated) to be capable of exerting enough force on scoring unit (140) to cause scoring element (140) to expand outward and into a body lumen (not shown). However the drug delivery membrane (610) may substantially conform to the surface of the scoring unit (140) while scoring unit (140) is being forced to expand due to pressure from dilatation balloon (120).

FIG. 1B shows a detail of the interface between the drug delivery membrane (610), the scoring element (710) formed by a scoring unit (140), and the outer wall (810) of dilatation balloon (120). Here scoring element (710) is simply a part of scoring unit (140), and the outer wall (810) is simply a part of dilation balloon (120).

In this detail, only a portion of the dilatation balloon (120) consisting of the outer skin, edge, or membrane of the balloon (810) is shown, along with, the hollow (usually fluid filled) interior of the dilatation balloon (830). The drug delivery membrane (610) and the outer skin of the dilatation balloon (810) will normally be separated by a gap (680), however in certain regions and in certain configurations, it may be useful to optionally join the drug delivery membrane (610) to the skin of the dilatation balloon (810) by occasionally bonding, gluing or welding the two materials together in selected regions (690). These bonded regions can be used to help control the relative configuration and positioning of the drug delivery membrane (610), the scoring element (710), and the dilatation balloon skin (810) as the dilatation balloon (120/810) expands and contracts.

In this configuration, drug delivery membrane (610) will typically be loaded with an appropriate therapeutic agent before use. The drug or therapeutic agent can either be embedded into or permeate the matrix of membrane (610), or alternatively the drug or therapeutic agent can be coated on the surface of membrane (610).

The catheter device (100) will be inserted in to an appropriate body lumen. Once it is in the desired (target) location, fluid (850) will normally be pumped in to the interior (830) of dilation balloon (120/810). This fluid will cause the dilatation balloon (120/810) to expand, moving the skin of the dilatation balloon (810) up against the scoring elements (710) of scoring unit (140). This pressure will cause scoring unit (140) to expand, and will move the scoring elements (710) up against the inner surface of drug delivery membrane (610). In this configuration, this pressure will cause drug delivery membrane (610) to distort and adhere to the scoring elements, forming membrane covered scoring elements (650). As a result, scoring element (710) effectively becomes coated with a layer (610) of material that contains therapeutic agents. Assuming that drug delivery membrane (610) has been chosen so as to be suitably thin, flexible, and strong, the scoring elements (710) will retain their ability to score a body lumen even when covered by the drug delivery membrane (610).

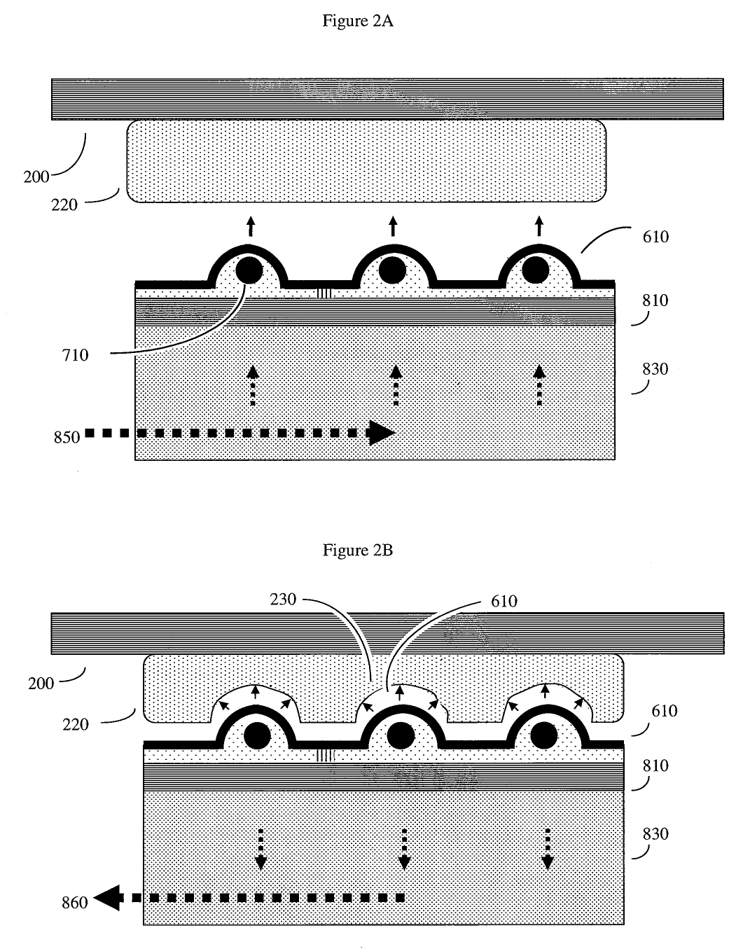

Reference now is made to FIG. 2A. In use, the catheter device (FIG. 1(100)) is inserted into the vascular system, for example, using a conventional catheter procedure, to a region of stenotic material (220) (e.g. plaque) on the lumen of a blood vessel (200). (The term “stenotic” is used herein to refer to the vascular lesion, e.g., the narrowed portion of the vessel that the balloon is meant to open.)

At the stenotic area (220), the dilatation balloon (120), (810)/(830), is inflated, for example, by liquid (850) flow into the balloon. The scoring elements (710) mounted on scoring unit (FIG. 1(140)) widens due to pressure from the inflated dilatation balloon. These scoring elements adhere against drug delivery membrane (610). On inflation, the dilatation balloon 120 together with the scoring elements (140), (710) and drug delivery membrane (610) are pressed against the walls of the blood vessel.

As shown in FIG. 2B, the pressure from the skin of the dilatation balloon (810) forces both scoring elements (710) and the drug delivery membrane (610) into the stenotic material (plaque) (220), causing stenotic material (220) to become both compressed and scored (230). Therapeutic agents (such as drugs) (620) flow by either direct contact or diffusion from the drug delivery membrane (610) (see small arrows) into the scored regions (230) of stenotic area (220). Some drug can flow to non-scored regions of the lumen as well.

After this dilatation and scoring step, dilatation balloon (FIG. 1(120), (810)/(830)) will normally be deflated by removing fluid (860) from the balloon (120) (810/830). Scoring elements (710) mounted on the scoring unit (FIG. 1(140) and drug delivery membrane (610) retract or narrow upon deflation.

Thus the dilatation device (FIG. 1(100)) is narrowed and may be readily retrieved from the blood vessel. The deflation profile of the deflated device (FIG. 1 (100)) is low and mainly circular. The stenotic material FIG. 2(220) in the blood vessel ideally remains pressed against blood vessel walls FIG. 2(220) to widen the available lumen and enhance blood flow.

In other embodiments, the scoring structure (140)/(710) of the present invention can have a non-helical configuration. Any design of a scoring structure that can accommodate an increase in the diameter of the balloon (FIG. 1(120)) upon inflation, and return to its configuration when the balloon is deflated, is a potentially appropriate design that may useful in the invention. In many embodiments, at least a portion of the scoring elements will not be parallel to the longitudinal axis of the balloon catheter to enhance flexibility and improve scoring.

Referring again to FIG. 1A, note that in some embodiments, scoring unit (140) is pushed outwardly by the inflation of the balloon (120), and is stretched by the inflation of the balloon. Thus when the balloon is deflated, scoring unit (140) may also assist in the deflation by its elastic recoil. This active deflation is faster, and also leads to a low profile of the deflated balloon. The balloon (120), disposed within the scoring unit (140), returns to its pre-inflated shape and forces the balloon to gain a low radial profile. Generally, drug delivery membrane (610) will be composed of a material that does not interfere with this inflation and deflation process.

In another embodiment of the invention, the catheter or dilatation device (100) may carry a stent. The stent can be crimped over the scoring unit (140). In this way, the scoring unit (140) can push the stent against hard areas of the lesion, enabling proper positioning of the stent against the vessel wall, even in hard-calcified lesions without pre-dilation.

In some embodiments, scoring unit (140) may have a helical structure that includes three wires that are attached to collars (150) and (160) at the proximal end and distal end, respectively. Alternatively the scoring structure may be formed as a metallic cage, which can be made from a slotted tube, or polymeric cage or polymeric external elements. Alternatively the scoring structure may comprise wires of other elements attached directly to the dilatation balloon material or close to the balloon ends.

If the scoring unit is made from nitinol wires, the diameter of the nitinol wires is typically in the range of 0.05 mm to 0.5 mm. Alternatively, a cage (for example a metallic cage made of a slotted tube) can be used in several configurations that allow local stress concentrations. The size and shape of the cross section of the cage elements or the cross section of the wires can vary. The cross section can be a circle, rectangle, triangle, or other shape.

In alternative embodiments, the nitinol wires may comprise short segments that are attached to the dilatation balloon (120).

In some embodiments, drug delivery membrane (610) is composed of a thin strong membrane material chosen to both capable of absorbing therapeutic agents, capable of deforming around the nitinol wire scoring elements (140), and pliable enough to not impart a significant resistance against the inflation and deflation of dilatation balloon (120) and helical unit (140). Drug delivery membrane (610) may be a nylon based material (polyamide), Pebax, or PET. The drug delivery membrane may be either a non-distensible material similar to the dilatation balloon material, a semi-compliant material, or an elastomeric compliant material.

Membrane materials may comprise a polymer matrix such as Pebax, polyurethane, rubber, polysulfone, nylon 11, nylon 12, ethylene-vinyl acetate copolymers, ethylene-acrylate ester copolymers, vinylpyrrolidone-vinyl acetate, styrene acrylic polymer, ethylene acrylic acid copolymer, carboxyl function acrylic polymer, hydroxyl function acrylic polymer, and acrylic dispersion polymer, among others. In some cases it is desirable to use a coherent bond coat (i.e. epoxies, acetals, acrylics, ethylene copolymers, or other suitable groups). Coatings may also comprise poly(glycol methacrylate), poly(methyl methacrylate), poly(ethyl methacrylate), poly(butyl methacrylate), poly(sulfanato ethyl methacrylate), poly(ethylene-co-vinyl acetate), poly(ethyl acrylate), poly(urethane-acrylate), poly(acrylamide-co-ethyl methacrylate), poly(divinyl benzene), poly(triethylene glycol-co-divinyl ether), poly(tri-methylol propane triacrylate), poly(pentaerythritol tetraacrylate), poly(bisphenol A ethoxylate diacrylate), poly(allyl ether), poly(diallyl maleate), poly(vinylidene fluoride), poly(triallyl isocyanurate), poly vinyl alcohol, ethylene vinyl alcohol copolymer, or alike. The drug may also be carried on the surface of the drug delivery membrane using an oxide layer or porous oxide layer. Alternatively the drug delivery membrane may be coated by drug without any polymer or carrying matrix of any kind.

The advantage of using a drug delivery membrane positioned over the scoring element is that more drugs can be delivered to the scored surface area and the adjacent tissue than could be delivered by just the scoring element alone. The drug delivery membrane's polymeric material may can load and transfer more drug than the nitinol surface of the scoring element. An additional advantage is that the polymeric material may disperse the cutting force of the coring elements. This could potentially result in less tissue cutting/trauma/damage, because the drug delivery membrane will distribute the scoring force over a larger area compared to the contact area of the uncovered nitinol scoring element contact surfaces.

When the device, properly sized to the target artery, is inflated, blood flow is occluded and nominal inflation pressure, or higher inflation pressure, of the device presses the membrane to the artery wall, thereby transferring drug to the artery wall tissue that is contacted by the drug coated membrane. Deflated, the drug delivery membrane refolds, similar to the underlining balloon.

If the drug delivery membrane made from a sufficiently elastic material, then when the inflation fluid is evacuated from the inner dilatation balloon, the elastomeric property of the drug delivery membrane helps it refold, thus functioning as an outer drug delivery balloon with no folds (zero-fold balloon). If the drug delivery membrane is not sufficiently elastic, then it still may be induced to refold properly when the underlying dilatation balloon is deflated if the drug delivery balloon is joined (glued, welded) to either the deflation balloon or the scoring structure at a plurality of separate points (e.g. spot welded in various locations) (See FIG. 1B (690). In this case the drug delivery membrane may function as a multiple-fold balloon in that it may present a wrinkled appearance when the dilatation balloon is deflated.

In addition to direct delivery to the scored region of the tissue, in this configuration, the drug delivery membrane may also deliver drug to surrounding regions of tissue that were not directly contacted by the scoring element. Drug absorbed by these surrounding tissues may also be useful for preventing restenosis from developing on the scored tissue.

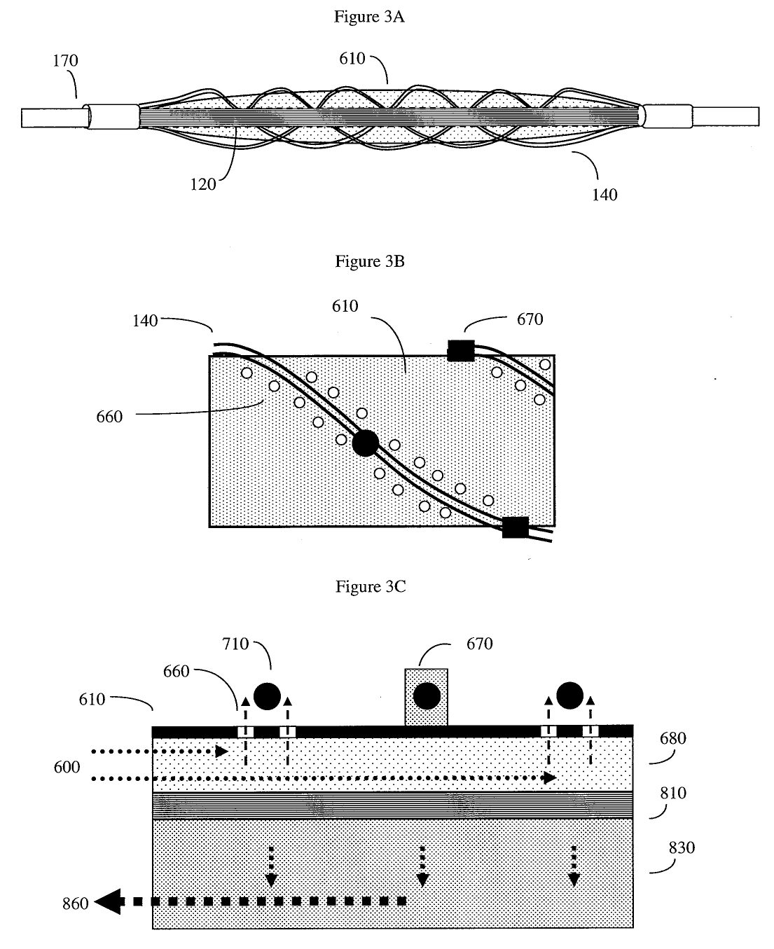

FIGS. 3A, 3B, and 3C show an alternative configuration in which the drug delivery membrane (610) is positioned on top of dilatation balloon (120) but underneath scoring structure (140). In this type of configuration, large volumes of therapeutic agents can be precisely delivered to the scored regions of the lumen. To do this, the drug delivery membrane (610) is perforated with numerous pores (660). The distribution of these pores can be determined by the device manufacturing process. In one embodiment, these pores may be preferentially distributed near the scoring elements (140).

If this configuration is desired, it may be also useful to ensure that the drug delivery membrane and the pores (660) maintain a proper orientation relative to the scoring elements (140) by welding or attaching the drug delivery membrane to the scoring elements at a plurality of contact points (670). This way, the pores will maintain their orientation relative to the scoring process throughout expansion and contraction of balloon (120).

In this embodiment, the proximal end of the catheter (170) may contain a first tube for inflating and deflating the dilatation balloon (120) by adding or withdrawing a fluid (850) and also a second tube for administering therapeutic agents (600) to the gap (680) between the drug delivery membrane (610) and the skin of the dilatation balloon (810), or to other drug delivery membrane drug reservoir.

FIG. 3B shows a close up of the outer surface of the structure shown FIG. 3A. In this configuration, drug delivery membrane (610) may contain a plurality of small pores (660) located near the various scoring elements (710). As previously discussed, to maintain a good dimensional relationship between the drug delivery membrane (610) and the scoring elements (710) as dilatation balloon (120)/(810) is inflated and deflated, in this embodiment drug delivery membrane (610) is shown intermittently attached to the scoring elements (710), (140), by a plurality of attachment sites (670).

FIG. 3C shows a detail of how this embodiment operates, focusing on the flow of dilation fluid and drugs. In this embodiment, the process of scoring and then applying drug to the scored regions of the lumen is a multi-step process.

In the processes taking place before the step shown in FIG. 3C, the dilation balloon fluid was applied to the dilation balloon (120), (810), (830), and the balloon pressure caused the scoring elements (710)/(140) to expand against a body lumen, scoring the plaque. This process was previously illustrated in FIG. 2A.

FIG. 3C shows that after the plaque has been scored, pressure in dilatation balloon may then be relaxed slightly by releasing (860) the dilation balloon fluid, while nearly simultaneously adding a liquid drug or therapeutic agent (600). With proper timing, therapeutic agent (600) will flow out through pores (660) and ideally deliver a high dose of therapeutic agents directly to the scored regions (e.g. plaque) (FIG. 2B (230)) of the body lumen. Because drug (600) can be applied from a reservoir located outside of the body if necessary, as much drug may be applied as deemed appropriate to the situation.

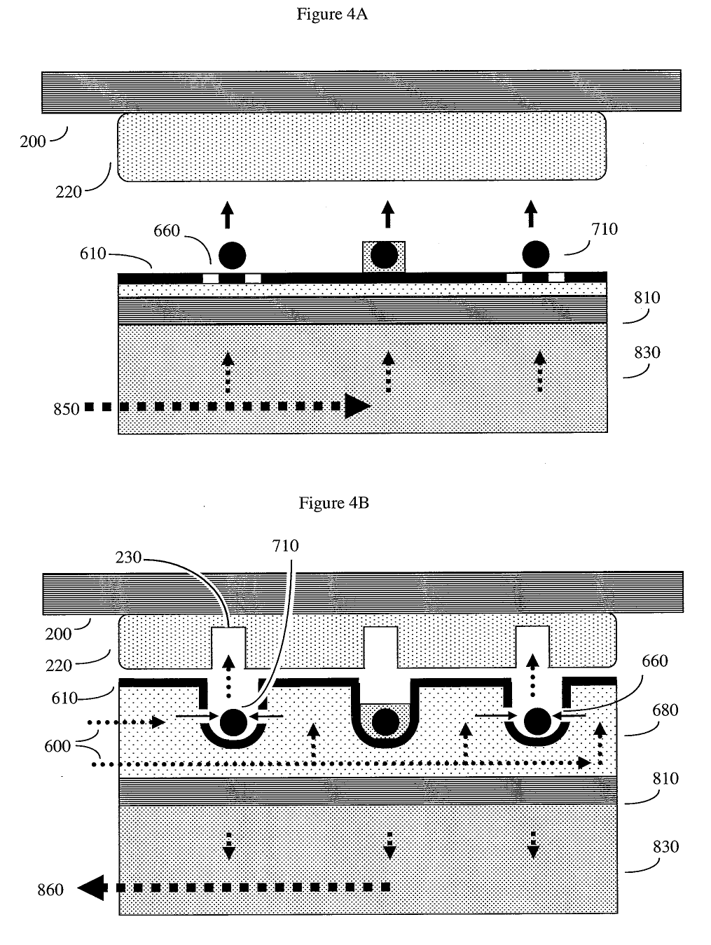

FIGS. 4A and 4B show an alternate embodiment of the invention. In this alternate embodiment, the drug delivery membrane (610) is chosen to be of a thin, elastic, and pliable material, such that the small amount of pressure caused by injected therapeutic agents (600) is enough to cause the drug delivery membrane to expand. However drug delivery membrane (610) is still not load bearing, and thus membrane (610) does not exert enough force to expand the scoring elements (710)/(140). Rather, drug delivery membrane (610) expands in between the scoring elements (710) (FIG. 4B).

Thus scoring elements (710)/(140), which don't expand due to the feeble pressure exerted by the drug delivery membrane (610), only expand due to the larger amount of pressure exerted by the dilation balloon (120)/(810). In this embodiment, the drug delivery membrane (610) may also be perforated by a plurality of pores (660), here again shown arranged near scoring elements (710).

In the example shown in FIG. 4A, the operator has inserted the catheter into an artery to a plaque occluded site, and is now expanding the dilation balloon (120)/(810) against a plaque target (220) on an artery lining (200) by injecting fluid (850) into the dilation/expansion balloon (120) (810), (830). Drugs (600) have not been injected yet. Thus drug delivery membrane (610) lies on top of the dilation balloon wall (810), and underneath the scoring elements (710). The force of the expanding dilation balloon wall (810) forces the scoring elements (710) up against and into the plaque (220).

In FIG. 4B, the scoring elements (710) have scored the plaque (220), creating scored regions (230), and the operator now wishes to treat the scored regions (230) with a drug or other therapeutic agent (600) immediately after the scoring process, and before the catheter is withdrawn from the body.

To do this, the infusion of the liquid therapeutic agent (600) is timed with a partial release of dilation balloon fluid (860). If the therapeutic agent (600) is injected at about the same rate at which the dilation balloon fluid (860) is removed, the overall outer volume of the inflated catheter head drug delivery membrane (810) remains roughly unchanged. However the inner dilation balloon (120), (810), (830), and the scoring elements (810) shrink. This volume is replaced by the expansion of the thin and pliable drug delivery membrane (610), which now expands in between the scoring elements (710) and somewhat replaces the lost volume. The drug delivery membrane (610) now contacts the plaque (220) on either side of the scored regions (230). This drug delivery membrane (610) acts to form a partial barrier to the entry of outside fluids to the scored area, and this same partial barrier acts to keep the injected therapeutic agent (600) localized to the scored area (230).

FIG. 4B shows this process in action. The scoring elements (710), now no longer supported by dilation balloon membrane (810), partially retract from the scored regions (230) of the plaque, exposing the scored regions. Therapeutic agents (600) are introduced to these scored regions (230) through pores (660) in the drug delivery membrane. The net result is that therapeutic agent (600) will highly localized to the scored regions (230), and will be delivered at a much higher concentration than might otherwise be possible.

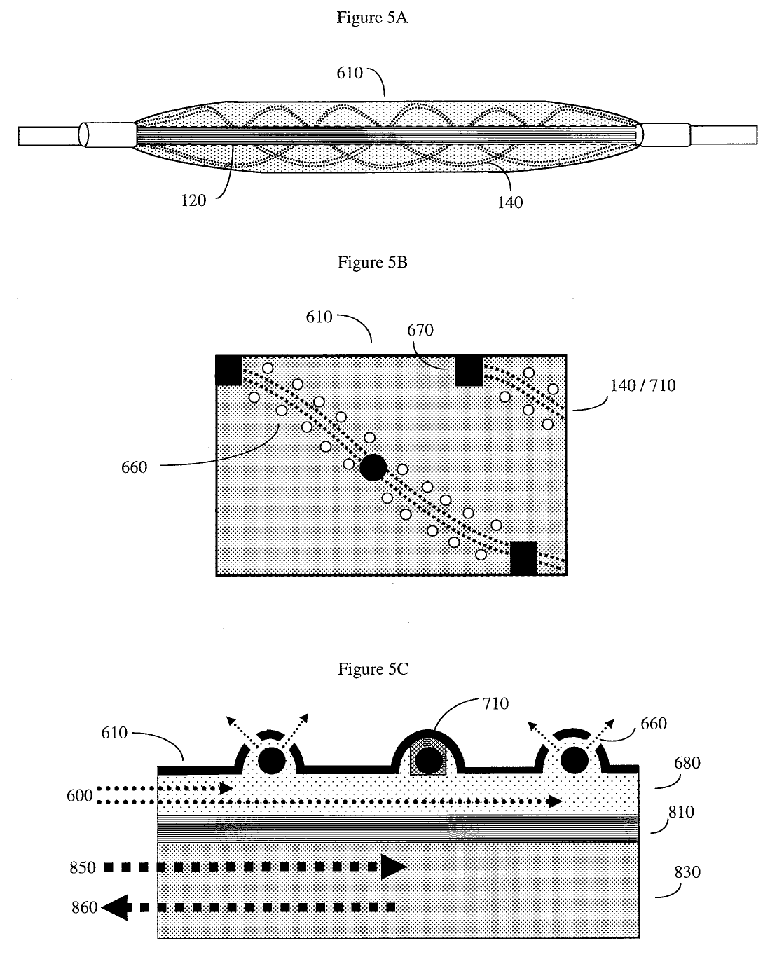

FIGS. 5A, 5B, and 5C show an alternative embodiment of the invention which combines concepts from FIGS. 1-2 and FIGS. 3-4. In this configuration, drug delivery membrane (610) is permeated by multiple pores (660), and is mounted outside of both the dilation balloon (120) and the scoring elements (140). An overview of this configuration is shown in FIG. 5A, and a close up of the pore configuration of this embodiment is shown in FIG. 5B.

As shown in 5B, the drug delivery membrane (610) is again permeated with a plurality of pores (660), which again may be manufactured to correspond to the distribution of the scoring elements (140/710) if this option is desired. As before, the alignment of the pores (660) and the scoring elements (140)/(710) may be maintained by a plurality of welds or spot adhesive regions (670) between the drug delivery membrane (610) and the scoring elements (140/710).

The operation of this embodiment is shown in FIG. 5C. The overall concept is similar to that previously shown in FIG. 2B, with the exception that in FIG. 2B, the drug delivery membrane (610) was pressed into the scored plaque regions (230) by the scoring elements, and the drug was released from membrane (610) by a more passive process such as direct contact or diffusion. By contrast, FIG. 5C shows a more active process in which the drug delivery membrane (610) has small pores (660), and drug (600) is actively pumped out of the pores and into the scored regions (FIG. 2B (230)).

As shown in 5C, the wall of the dilation balloon (810) is first inflated by fluid (850), pressing the scoring elements (710) into plaque (not shown), and creating a scored region of plaque (not shown, please see FIG. 2B which shows a similar configuration). After the scoring process, some of the balloon fluid is released (860) from the dilation balloon. At approximately the same time, the liquid drug or therapeutic agent (600) may be pumped into the space between the drug delivery membrane (610) and the dilation balloon (810). This drug (600) may be released from the drug delivery membrane (610) by pores (660) which, as previously shown in FIG. 5B, can be located near the scoring elements (710). This embodiment again delivers drug (600) at or near the scored regions of the plaque.

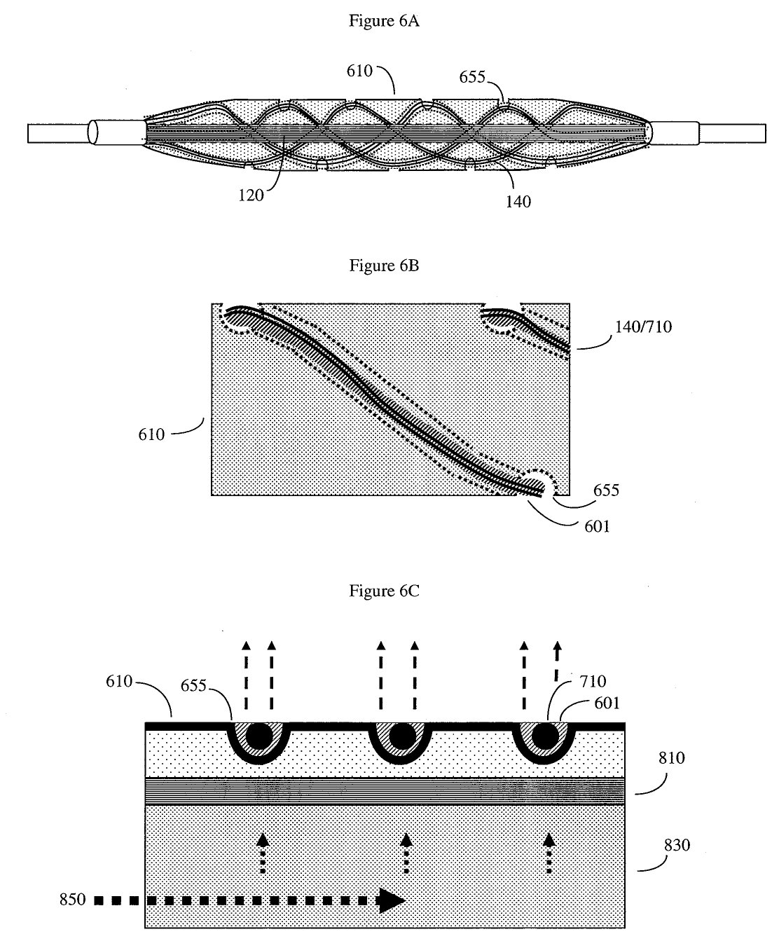

FIGS. 6A, 6B and 6C show an alternative embodiment of the invention in which the drug is stored in a semi-dry, polymer-mixed, or hydrogel form (601) in crevices (655) of the drug delivery membrane (610). These crevices are formed when the drug delivery membrane (610) wraps, wrinkles, or is deformed around the various scoring elements (140/710). An overview of this embodiment is shown in FIG. 6A.

FIGS. 6B and 6C show the details of this embodiment. In this embodiment, typically drug delivery membrane (610) is not elastic. Although the drug delivery membrane is nominally mounted inside of scoring cage or elements (140/710), it typically will be a multiple fold membrane with enough excess surface area in the collapsed configuration to expand along with dilation balloon (120/810) when the dilation balloon is expanded by fluid (850).

Here, drug delivery membrane (610) is configured so that in when the dilation balloon (120/810) is in the deflated form, the scoring elements (140/710) are stored in the folds (655) of the drug delivery membrane, along with an appropriate drug or therapeutic agent (601), here normally stored in a partially immobilized form, such as a solid, semi-solid, polymeric delivery system, or hydrogel form.

As shown in FIG. 6C, when the scoring elements (710) expand due to force from the dilation balloon fluid (850) and dilatation balloon wall (810), the therapeutic agent (601), carried by membrane folds (655) is administered to the scored area of the plaque (see FIG. 2B (230) at the same time the scoring element (710) scores the plaque.

The methods and systems of the present invention are particularly useful for delivering drugs which are hydrophobic and lipophilic. These drugs are often difficult to distribute precisely because they are difficult to dissolve in aqueous media, and also tend to stick to non-target sites such as other lipid containing body components. However when delivered precisely to the target using the devices and methods of the present invention, the hydrophobic nature of some drugs (e.g. paclitaxel and sirulimus) and the fact that those drugs are lipophilic (i.e. high affinity to cell membranes and liposomes) help retain the drug for longer time in the target area. This minimizes the loss of target-site delivered drug during and after the time of delivery due to dissolution in the blood.

In particular examples, a resorbable or non-resorbable polymer matrix may first be applied on at least a portion of an exposed surface of the drug delivery membrane, and the drug later absorbed into a porous structure of the drug delivery membrane polymer carrier matrix.

The polymer matrixes in the scoring catheter associated membrane system used to deliver the drug may often have properties that are different from the polymer matrixes used to deliver drugs for drug eluting stents. Whereas drug eluting stents are designed to release drugs over a period of days or weeks, the drug delivery membranes of the present invention are often designed to release drug over a very short period of time, such as a few seconds to a few minutes. Thus, in contrast to drug eluting stents, the present membrane delivery system may utilize both rapidly dissolving polymers, and rapid drug release polymers.

Suitable polymeric carriers may be resorbable, such as those comprising polylactic acids (PLA), polyglycolic acids (PLG), collagens, and the like. Alternatively, the polymeric carrier may be a porous but non-resorbable material such as porous silicon or polyethylene. Hydrogels such as Poly Ethylene Oxide (PEO) may be used and release the drug through swelling and erosion. Degradable polymers which include polyhydroxyalkanoate can be used as well. The polymer can coat the scoring element struts or alternatively can create a film between at least some of the scoring element struts or any combination of the above.

In some cases, it may be advantageous to choose a drug delivery membrane polymer and structure from materials that to allow drugs to penetrate and elute more quickly when the material has an expanded structure, but retard such drugs from penetrating or eluting when the material has a less expanded structure. Here this property can be particularly useful, because such drug eluting membranes will thus tend to hold on to the drugs while the catheter is being introduced to the target zone and the underlying balloon is deflated, yet tend to rapidly release the drugs at the correct target zone. This is because at the correct target zone, the operator will expand the underlying balloon, and this balloon in turn will expand the drug delivery membrane to a wider diameter. The structure of the drug carrying material in the drug delivery membrane will also expand, and the drug will be released more rapidly.

This membrane system can deliver a very wide variety of different active substances including drugs useful for treating many different luminal diseases and conditions. Some of the many drugs, therapeutic, and pharmaceutical agents and active substances that may be delivered by the present invention are discussed below:

(1) antiproliferative and antimitotic agents such as natural products such as vinca alkaloids (i.e. vinblastine, vincristine, and vinorelbine), paclitaxel, epidipodophyllotoxins (i.e. etoposide, teniposide), antibiotics (dactinomycin, actinomycin D, daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine);

(2) antiplatelet agents such as G(GP) II.b/III.a inhibitors and vitronectin receptor antagonists;

(3) alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC);

(4) antiproliferative and antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine});

(5) platinum coordination complexes such as cisplatin, carboplatin, procarbazine, hydroxyurea, mitotane, and aminoglutethimide;

(6) hormones (e.g. estrogen);

(7) anticoagulants (heparin, synthetic heparin salts and other inhibitors of thrombin);

(8) fibrinolytic agents (such as tissue plasminogen activator, streptokinase and urokinase), aspirin, dipyridamole, ticlopidine, clopidogrel, abciximab;

(9) antimigratory agents;

(10) antisecretory agents (breveldin);

(11) anti-inflammatory agents, such as adrenocortical steroids (cortisol, cortisone, fludrocortisone, prednisone, prednisolone, 6.alpha.-methylprednisolone, triamcinolone, betamethasone, and dexamethasone), non-steroidal agents (salicylic acid derivatives i.e. aspirin; para-aminophenol derivatives i.e. acetaminophen;

(12) indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate);

(13) immunosuppressive agents such as cyclosporine, tacrolimus (FK-506), sirolimus (rapamycin), azathioprine, mycophenolate, mofetil;

(14) angiogenic agents such as vascular endothelial growth factor (VEGF), fibroblast growth factor (FGF);

(15) angiotensin receptor blockers;

(16) nitric oxide donors;

(17) anti-sense oligionucleotides and combinations thereof;

(18) cell cycle inhibitors, mTOR inhibitors, and growth factor receptor signal transduction kinase inhibitors;

(19) retenoids;

(20) cyclin/CDK inhibitors;

(21) HMG co-enzyme reductase inhibitors (statins); and

(22) protease inhibitors.

Additionally, nucleic acid reagents, such as viral gene vectors, antisense agents, may be used, or alternatively living cells, such as in-vivo modified stem cells, fibroplasts, myoblasts, satellite cells, pericytes, cardiomyocytes, skeletal myocytes, and macrophages may be used.

In another embodiment, antibodies, particularly monoclonal antibodies, designed to bind to targets involved in restenosis (e.g. against smooth muscle, coagulation, tissue factor, or platelets) may also be used.

In addition to therapeutic drugs, diagnostic agents such as radio-opaque X-ray contrast agents, magnetic resonance imaging (MRI) dyes (contrast agents), or ultrasound contrast agents may be used to help visualize the status of the lumen. Alternatively, chromogenic, fluorescent, or luminescent dyes (contrast agents) may also be used.

There are various ways to manufacture the device. One way is to wrap or fold the drug delivery membrane over the drug scoring elements, and then bond the membrane to these drug scoring elements. Alternatively, the drug delivery membrane can be made of a elastic balloon like material (here Pellethane or other thermoplastic elastomers may be used), and this elastic drug delivery membrane can then be bonded to the proximal and distal catheter ends at or near the positions where the dilation balloon is bonded.

Alternatively, the drug delivery membranes may be formed from extruded material by an air or vacuum blowing process, much as balloons are manufactured. Here bonding tabs would be incorporated at the distal and proximal end of the blown tubes. Typically, the wall thickness of these membranes will be approximately 0.0005″ to 0.0020″ thick.

Note however that this balloon blowing process will not normally produce macroscopic pores in the drug delivery membranes. Thus if such pores are desired for drug delivery purposes (as will be described in later examples) these pores may be created by a laser cutting, photochemical etching, or some other perforation method in a later manufacturing step.

This balloon shaped drug delivery membrane can then be mounted on top of the scoring element structure, and optionally bonded in selected regions to portions of the scoring elements and the catheter. In some embodiments, the drug delivery membrane can then be coated with the drug, or a drug polymer combination.

There are various ways to load the drug onto the drug delivery membrane. In one method, the underlying balloon is expanded, drug or drug polymers applied to the drug delivery membrane by spray coating or other process, volatile portions of the drug or drug polymer mix optionally allowed to dry, and the underlying balloon then deflated. In addition to spray coating, the drugs and other active substances can be applied to one or more surface regions of drug delivery membrane by other conventional techniques, such as dipping, painting, vapor deposition, spin coating, and the like.

The active substances may be applied in an essentially pure form, i.e., in the absence of any carriers, diluents, adjuvants, modifiers, enhancers, or the like. More commonly, however, the active substances will be applied with or combined into a suitable carrier, matrix, or other chemical structure which can facilitate or control release of the drug over a desired time period or immediately upon contact of the drug delivery membrane to the body lumen. For example, the substance(s) may be present in a biodegradable or bioresorbable matrix such as a polymeric nanoparticle or a polymeric microparticle.

In other embodiments of the invention, the scoring unit (140) may be glued, thermally bonded, fused or mechanically attached at one or both ends to dilatation balloon (120). Alternatively the drug delivery membrane (610) may be glued, thermally bonded, fused, or mechanically attached to either the helical unit (scoring element), or the dilatation balloon at a plurality of locations.

Many alternate scoring structures may be used. The scoring structure may comprise wires that are attached to the dilatation balloon in helical configuration or other configuration. The wires may be thermally attached to the dilatation balloon or glued, mechanically attached, or the like. The scoring structure may also comprise wire or cage elements that are not parallel to the longitudinal axis of the dilatation balloon so that the combination of the scoring structure and the dilatation balloon remains flexible.

The scoring structures may be attached directly to the balloons or other shells, in some cases being embedded in the balloon material, but will more usually be formed as separate cage structures which are positioned over the balloon and attached to the catheter through attachment elements on either side of the balloon.

The expandable cages may be formed using conventional medical device fabrication techniques, such as those used for fabricating stents, such as laser cutting of hypotube and other tubular structures, EDM forming of hypotubes and tubes, welding of wires and other components and the like.

Typically, such expandable shell structures will comprise the attachment elements and an intermediate scoring section between the attachment elements. As illustrated in the embodiments above, the attachment elements may be simple cylindrical or tube structures which circumscribe the catheter body on either side of the balloon or other expandable shell. The simple tube structures may float over the catheter body, i.e., be unattached, or may be fixed to the catheter body. A number of alternative embodiments for the attachment elements will be described in connections with the embodiments below.

The intermediate scoring sections may also have a variety of configurations where at least some of the scoring elements will typically be disposed in a non-axial configuration, i.e., in a direction which is not parallel to the axial direction of the expandable cage. A preferred configuration for the intermediate scoring section comprises one or more helical elements, generally as illustrated in the prior embodiments. Other exemplary configurations are set forth in the embodiments described below.

Generally, expandable cage structures, such as a helical version of scoring element (140) will be mounted over a dilatation balloon (120) with the attachment elements secured to the catheter body on either side of the dilatation balloon. The tube or cylindrical attachment elements (150), (160) may simply float over the catheter body. In other embodiments, however, it may be desirable to use an adhesive or other means for affixing either one or both of the attachment elements to the catheter body. Having at least one floating attachment element, however, is often desirable since it can accommodate shortening of the intermediate scoring section as that section radially expands. In other cases, however, the individual scoring elements may possess sufficient elasticity to accommodate such shortening. For example, nitinol and other shape memory alloys possess significant stretchability, typically on the order of 8% which in some instances will be sufficient to accommodate any tension applied on the intermediate scoring section by radial expansion of the balloon.

The compliance of the system may be adjusted by varying any one or combination of material, wall thickness, or length of the scoring structure, balloon, or drug delivery membrane. The catheter tube used to deliver the device may comprise any elastomer, such as elastic polymer like Nylon, Pebax, or PET. Typically, the catheter tube is formed from extruded tubing, but may also comprise braided polymeric or metallic fibers, or wire mesh. A high memory metal such as nitinol or stainless steel may also be used.

In some embodiments, the compliance of the scoring structure (140), dilatation balloon (120), and optionally drug delivery membrane (610), is controlled by actuating a manipulator during expansion or contraction of the radially expandable shell. In one aspect, an attachment structure may be axially advanced with respect to the catheter body as the dilatation balloon is being inflated or deflated. For example, an attachment structure may be pushed toward the distal end of the catheter body while the dilatation balloon is being expanded, to constrain the compliance of the dilatation balloon. An attachment structure may also be pulled away from the distal end of the catheter body during or after the dilatation balloon is being deflated to minimize the profile of the balloon and scoring structure. Alternatively, a manipulator (not shown) may be used to rotate an attachment structure with respect to the catheter body to control the compliance of the dilatation balloon and scoring structure during transition from a collapsed to expanded state and back to a collapsed state.

In addition to manipulating the configuration of the dilation balloon and the scoring structure, the drug delivery membrane and or the delivery of therapeutic agents may also be under operator control during this procedure. This control may be accomplished by various mechanisms on the proximal end of the catheter tube, such as various pumps, switches, motors, control rods, shafts, wires, and the like.

What is claimed is:

1. A system for treating a vascular wall, said system comprising:
a catheter including a catheter body having a proximal end and a distal end;
an expandable scoring structure disposed at a distal end of the catheter;
a membrane adapted to be positioned over at least a part of the expandable scoring structure; and
a substance sequestered on or in the membrane, wherein at least a portion of the substance is released from the membrane when the scoring structure is expanded within a body lumen.

2. A system as in claim 1, wherein the expandable scoring structure comprises an inflatable balloon.

3. A system as in claim 2, wherein the expandable scoring structure further comprises a cage disposed over the balloon, wherein the cage opens with the balloon inflation and resiliently closes over the balloon upon deflation.

4. A system as in claim 1, wherein the membrane is disposed at least partially over the cage structure.

5. A system as in claim 1, wherein the membrane is disposed at least partly between the cage structure and the balloon.

6. A system as in claim 1, wherein the membrane has a tubular geometry shaped to be disposed over the expandable scoring structure.

7. A system as in claim 1, wherein the membrane comprises a sheet adapted to be wrapped over the expandable scoring structure.

8. A system as in claim 1, wherein the membrane is elastic so that it expands and contracts as the balloon is inflated and deflated.

9. A system as in claim 1, wherein the membrane is non-distensible or semi-compliant and folded over the balloon before the balloon is inflated.

10. A system as in claim 1, wherein the substance is selected from the group consisting of antiproliferative agents, antimitotic agents, antiplatelet agents, alkylating agents, platinum coordination complexes, hormones, anticoagulants, fibrinolytic agents, antimigratory agents, antisecretory agents, anti-inflammatory agents, indole acetic acids, indene acetic acids, immunosuppressive agents, angiogenic agents, angiotensen receptor blockers, nitric oxide donors, anti-sense oligonucleotides, cell cycle inhibitors, mTOR inhibitors, growth factor receptor signal inhibitors, transduction kinase inhibitors, retenoids, cyclin/CDK inhibitors, HMG co-enzyme reductase inhibitors, protease inhibitors, viral gene vectors, stem cells, in-vivo modified stem cells, macrophages, monoclonal antibodies, paclitaxel, sirulimus, x-ray contrast agents, MRI contrast agents, ultrasound contrast agents, chromogenic dyes, fluorescent dyes, and luminescent dyes.

11. A system as in claim 10, wherein the substance is present in a biodegradable or bioresorbable matrix selected from the group consisting of polymeric nanoparticles and polymeric microparticles.

12. A system as in claim 1, wherein the membrane comprises a polymer.

13. A system as in claim 12, wherein the polymer is selected from the group consisting of Pebax, polyurethane, rubber, polysulfone, nylon 11, nylon 12, ethylene-vinyl acetate copolymers, ethylene-acrylate ester copolymers, vinylpyrrolidone-vinyl acetate, styrene acrylic polymer, ethylene acrylic acid copolymer, carboxyl function acrylic polymer, hydroxyl function acrylic polymer, acrylic dispersion polymer, poly(glycol methacrylate), poly(methyl methacrylate), poly(ethyl methacrylate), poly(butyl methacrylate), poly(sulfanato ethyl methacrylate), poly(ethylene-co-vinyl acetate), poly(ethyl acrylate), poly(urethane-acrylate), poly(acrylamide-co-ethyl methacrylate), poly(divinyl benzene), poly(triethylene glycol-co-divinyl ether), poly(tri-methylol propane triacrylate), poly(pentaerythritol tetraacrylate), poly(bisphenol A ethoxylate diacrylate), poly(allyl ether), poly(diallyl maleate), poly(vinylidene fluoride), poly(triallyl isocyanurate), poly vinyl alcohol, ethylene vinyl alcohol copolymer, collagen, polyethylene, poly ethylene oxide, and polyhydroxyalkanoate.

14. A system as in claim 11, wherein the polymer matrix is permeable and the substance is absorbed therein.

15. A system as in claim 11, wherein the substance is coated on a surface of the membrane.

16. A system as in claim 11, wherein the polymer is dissolvable in the vascular environment.

17. A system as in claim 15, wherein the drug is dispersed within the polymer and released as the polymer dissolves in the vascular environment.

18. A method for treating a luminal wall, said method comprising:
positioning an expandable scoring structure at a treatment site in a body lumen of a patient;
expanding the scoring structure so that said scoring structure scores a region of or on the luminal wall; and
releasing a substance into the scored material from a membrane disposed as least partially over the expandable scoring structure.

19. A method as in claim 18, wherein the scoring structure scores occlusive material on a vascular wall so that the substance is released into the occlusive material.

20. A method as in claim 18, wherein the scoring structure scores the vascular wall so that the substance is released into the vascular wall.

21. A method as in claim 18, wherein the substance is selected from the group consisting of antiproliferative agents, antimitotic agents, antiplatelet agents, alkylating agents, platinum coordination complexes, hormones, anticoagulants, fibrinolytic agents, antimigratory agents, antisecretory agents, anti-inflammatory agents, indole acetic acids, indene acetic acids, immunosuppressive agents, angiogenic agents, angiotensen receptor blockers, nitric oxide donors, anti-sense oligonucleotides, cell cycle inhibitors, mTOR inhibitors, growth factor receptor signal inhibitors, transduction kinase inhibitors, retenoids, cyclin/CDK inhibitors, HMG co-enzyme reductase inhibitors, protease inhibitors, viral gene vectors, stem cells, in-vivo modified stem cells, macrophages, monoclonal antibodies, paclitaxel, sirulimus, x-ray contrast agents, MRI contrast agents, ultrasound contrast agents, chromogenic dyes, fluorescent dyes, and luminescent dyes.

22. A method as in claim 18, wherein expanding comprises inflating a balloon having a scoring cage thereon.

23. A method as in claim 22, wherein the membrane is disposed over the scoring cage.

24. A method as in claim 22, wherein the membrane is disposed between the scoring cage and the balloon.
